(19) United States
(12) Patent Application Publication
(10) Pub. No.: US 2016/0200500 A1
(43) Pub. Date: Jul. 14, 2016

(54) L-Ascorbic Acid Formulations and Methods of Use for Skin Care

(71) Applicant: Lavico Inc., East Windsor, NJ (US)

(72) Inventor: Spyridon A. Foteinos, Kifissia - Attica (GR)

(21) Appl. No.: 14/989,312

(22) Filed: Jan. 6, 2016

Related U.S. Application Data
(60) Provisional application No. 62/100,513, filed on Jan. 7, 2015.

(51) Int. Cl. B65D 81/32; B65D 51/24; A61Q 19/00; A61K 8/67; A61Q 19/08

(52) U.S. Cl. CPC B65D 81/3277; A61K 8/676; A61Q 19/08; A61Q 19/00; B65D 51/24; A61K 2800/882; A61K 2800/87

(57) ABSTRACT

L-ascorbic skin treatment formulations are provided in a storage container in the form of a vial construction having separate first and second compartments. Solid L-ascorbic acid is located in the first compartment and a liquid vehicle composition is located in the second compartment. The vial is constructed to allow for the combining of the L-ascorbic acid and the liquid vehicle composition by a user immediately prior to a topical application of the combined formulation to the skin. The first compartment may be in the form of a plug for the vial. A cover may be provided which, when screwed down, punctures a wall of the first compartment to allow the mixing of the contents of the two compartments.

### BACKGROUND OF THE INVENTION

Ascorbic acid, and in particular L-ascorbic acid, a form of vitamin C, is recognized as having potent antioxidant properties. Thus it is of significant skin care value, and provides photoprotection, skin lightening, wound healing, and skin rejuvenation properties. Its critical role in collagen synthesis leads to an intense anti-wrinkle/anti-ageing result, further establishing it as a vital molecule for skin health.

Since humans lack one of the final enzymes in the biosynthesis of ascorbic acid, it is not synthesized in the body, so humans are dependent on external dietary sources to satisfy the body's needs. Thus the food, dietary supplements and topical preparations containing L-ascorbic acid or vitamin C have beneficial effects on skin cells. An important link in the chain is vitamin C's transport.

After a topical application, the skin's stratum corneum layer is the primary obstacle to efficient vitamin C absorption. It's critical for an applied Vitamin C formula to overcome as much as possible of this barrier by optimizing the pH and concentration of the applied solution, along with use of appropriate enhancers. As vitamin C in solution degrades over time, an important factor in maintaining efficacy of a topically applied vitamin C product is assuring that the vitamin C has not degraded during storage and prior to application.

### BRIEF DESCRIPTION OF THE INVENTION

Accordingly, the present invention is a skin treatment system and a method of treatment utilizing vitamin C, and particularly its L-ascorbic acid form, as an active constituent that keeps the vitamin C in a condition that is not prone to deterioration prior to use. In particular, the invention incorporates a vial dispenser construction that keeps the vitamin C in a solid form separated from the rest of the formulation as long as the product is on the shelves and until the time of use. Just before the use, the vitamin C is combined or mixed with the liquid vehicle containing other constituents to disperse it thoroughly. The product is then to be used within a short time interval as defined by relevant stability considerations.

### BRIEF DESCRIPTION OF THE DRAWING

A fuller understanding of the invention will be realized upon consideration of the following description of a preferred illustrative embodiment of the invention, when taken in association with the annexed

FIG. 1 which presents a view of a vial system of the invention.

### DETAILED DESCRIPTION OF THE INVENTION

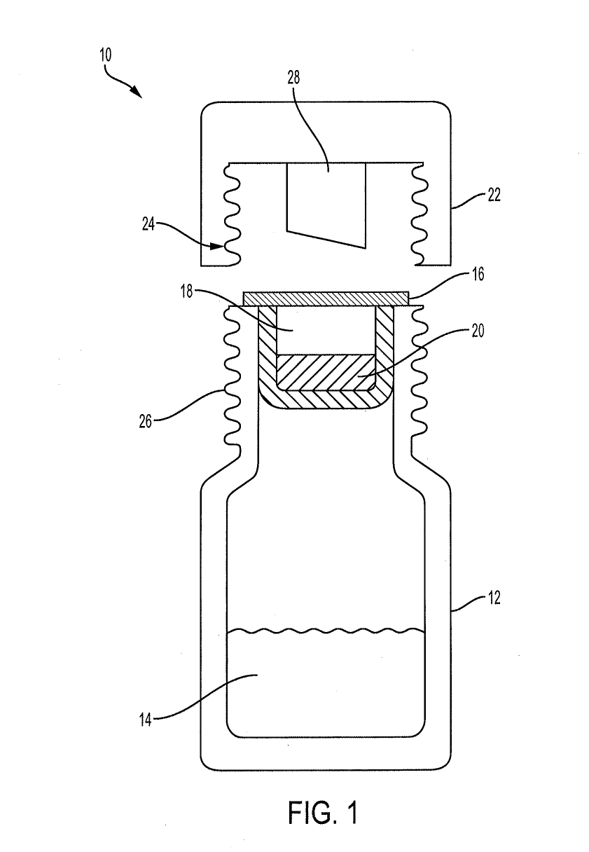

FIG. 1 depicts a vial system 10 which may be used in connection with the invention. It includes a main body chamber 12, in which the liquid composition 14 of non-sensitive components is located. The composition includes a liquid vehicle, such as water, that is compatible with the vitamin C and which allows the vitamin C to fully disperse through the composition. The vial is closed by seal 16, which has a well 18 in which the vitamin C, 20, is located. Cap 22 has an interior thread 24 to mate with the vial's exterior thread 26 and has plunger 28 adapted to pierce the lower end of well 18 when the cap is screwed downwardly upon the vial. The seal, or at least the bottom well wall thereof, is constructed of a material able to be pierced by the bottom face 30 of the plunger as it is depressed, and thus may be, for example, of a thin plastic. The bottom face 30 of the plunger is constructed to efficiently puncture and preferably separate the well bottom wall from the remainder of the seal as and thus may be canted as depicted or otherwise provided with protrusions or the like to assist in the puncture action. With the dislodgement or puncture of the seal, the vitamin C drops through the created opening into the vial to mix with the composition 14. The vial may be shaken to effect complete blending. The cap can then be removed from the vial and an applicator cap installed to dispense the blended composition outwardly through the upper end of the vial.

Preferred compositions for use in the present invention contain L-ascorbic acid in an amount of from 2% to 25% by weight. The composition 14 may comprise a humectant, such as an alkanediol, wherein the alkanediol is propanediol, also known as propylene glycol, and in particular, 1,2-propanediol. The alkanediol may also be 1,3-butanediol, 1,2-butanediol, or 1,2-ethanediol, among others.

The alkanediol may be present in the overall composition, including the added L-ascorbic acid, at 10% to 40% by weight. In a preferred embodiment, the alkanediol comprises 25% of the composition.

Preservatives having antibacterial activity are optionally present in the compositions. Any preservative commonly used in cosmetic formulations is acceptable, such as potassium sorbate, diazolidinyl urea, etc.

Moisturizers are also optionally present. Any moisturizer commonly used in cosmetic formulations may be an acceptable, and include glycerin, hyaluronic acid, sodium hyaluronate and the like.

In addition, a viscosity enhancer, such as hydroxyethylcellulose is also optionally present in the present invention, and may be incorporated in a range of 0.1-0.6% by weight.

Other components may be optionally included to enhance the properties of the product. Such components may fall into several functional classes, including penetration enhancers such as dimethyl isosorbide, oleic acid and linoleic acid; firming components, such as chicory root oligosaccharides and pullulan; lines and wrinkle optical “minimizers”, such as poly(methyl methacrylate), matrixyl, oligopeptides, and mu-conotoxin, as sold as XEP-018™ by Activen SA and Covabead Velvet™ by Sensient Cosmetics Technologies. In addition, other components, such as dimethyl isosorbide (DMI) may be included to enhance skin permeation of the functional ingredients.

### EXAMPLE 1

### Ascorbic Acid Composition 1

The liquid composition comprises water, propylene glycol and hydroxyethylcellulose in the following respective ratios (by weight): 70-75%; 20-30% and 0.1-0.8 mixed together. To this mixture preservatives, such as potassium sorbate and diazolidinyl urea in a 1:1 weight ratio, are added. The L-ascorbic acid is separately provided in a weight percentage of 10% of the overall composition is separately provided.

### EXAMPLE 2

### Ascorbic Acid Composition 2

The liquid composition comprises water, propylene glycol, and hydroxyethylcellulose in the following ratios: 70-75%; 20-30%; and 0.1-0.8% mixed together. To this mixture a preservative, such as potassium sorbate and diazolidinyl urea in a 1:1 ratio, is added. Moisturizers such as hyaluronic acid in the range of 0.001-0.01% and glycerin in the range of 1.0-2.0% by weight are then added to the formulation. The L-Ascorbic acid is separately provided to yield a weight percentage of 10% to the overall composition is separately provided.

### EXAMPLE 3

### Ascorbic Acid Composition 3

The liquid composition comprises water, propylene glycol and hydroxyethylcellulose in the following ratios: 70-75%; 20-30%; and 0.1-0.8% mixed together. To this mixture a preservative, such as potassium sorbate and diazolidinyl urea in a 1:1 ratio, is added. A moisturizer, such as hyaluronic acid in a range of 0.001-0.01% and glycerin in the range of 1.0-2.0% by weight is also added to the formulation. The L-ascorbic acid is separately provided to yield a weight percentage of 10% of the overall composition. A pH-adjustment (neutralizing) agent such as sodium hydroxide, potassium hydroxide, or L-arginine may also be added to the composition as appropriate.

A statistical analysis report of a clinical trial with two “therapeutic” stages, one of 30 days duration and, the other one of 90 days duration, in which the formulation of Example 1 dispensed immediately upon mixing the L-ascorbic acid with the other constituents on women aged over 35 years, accompanied by tests to assess cutaneous tolerance, sensitization and photosensitization, yielded the following:
a. Wrinkle depth evaluation: depth reductions of 12% & 26.4%, respectively, were encountered after the 30 & 90 days treatment periods.
b. Improvement in skin texture by 26% & 52%, after 30 & 90 days, respectively.
c. Improvement in skin glossiness by 35% & 65%, after 30 & 90 days, respectively.
d. Pigmentation disorder improvement (decrease) by 12% & 30%, after 30 & 90 days, respectively

The product was highly rated by the users participating in the study (153 women), with very positive comments on various attributes. The scores were between 4.1-4.6 (in a range of 0-5). As an example, the score for “a good opinion about the product” was 4.6.

Safety was also confirmed on 40 healthy volunteers, according to which the product can be considered hypoallergenic.

### EXAMPLE 4

### Ascorbic Acid Composition 4

The liquid composition comprises water, propylene glycol, DMI, XEP-018 and Covabead Velvet 10 & 20. The L-ascorbic acid is separately provided to yield a weight percentage of 10-25%.

I claim:

1. The combination of an L-ascorbic skin treatment formulation and storage container therefor, comprising a vial construction having separate first and second compartments, solid L-ascorbic acid in the first compartment and a liquid vehicle composition in the second compartment, the vial being further adapted and constructed to allow for the combining of the L-ascorbic acid and the liquid vehicle composition by a user immediately prior to a topical application of the combined formulation to the skin.

2. The combination of claim 1, wherein the L-ascorbic acid is present in the combined formulation at a weight percentage of from 2 to 25 percent.

3. The combination of claim 2, wherein the liquid vehicle comprises at least one of a moisturizer, preservative, and viscosity enhancer.

4. The combination of claim 1, wherein the vial construction comprises a necked vial forming the second compartment, the first compartment forming a closure for the second compartment and being located in the vial neck and having a bottom wall separating the first and second compartments; and further comprising a cover for the necked vial having means for penetrating the bottom wall to permit the contents of the two compartments to intermix.

5. The combination of claim 4, wherein the vial neck has an external thread and the cover has a mating internal thread, the penetrating means being located and arranged on an inner surface of the cover whereby screwing the cover down upon the neck of the vial from a first position wherein the penetrating means is located above the first compartment to a second position causes the penetrating means to penetrate the wall of the first compartment.

6. The combination of claim 4, wherein the first compartment has a top wall, the cover first position positions the penetrating means above the top wall.

7. A method for the application of L-ascorbic acid to the skin, comprising the steps of:
providing a dispenser having a first chamber holding solid L-ascorbic acid and an adjacent second chamber holding a liquid vehicle for the L-ascorbic acid;
breaking a wall of the first chamber such that the L-ascorbic acid and the liquid vehicle can mix;
allowing the L-ascorbic acid to be substantially dissolved in the vehicle; and
immediately applying the vehicle and dissolved L-ascorbic acid to the skin.

8. The method of claim 7 wherein the wall to be broken of the first chamber is part of a seal for the second chamber and the step of breaking the wall of the chamber is performed by screwing a cap down upon the second chamber.

9. The method of claim 8 wherein the cap has a downwardly extending interior projection which penetrates the wall to be broken when the cap is screwed down.

10. The method of claim 7 further comprising the step of removing the cap from the second chamber after the L-ascorbic acid is mixed with the liquid vehicle and affixing an applicator thereto prior to applying the vehicle and dissolved L-ascorbic acid to the skin.
